(19) United States
(12) Patent Application Publication
(10) Pub. No.: US 2016/0089511 A1
(43) Pub. Date: Mar. 31, 2016

(54) Device for reducing the carbon dioxide concentration of inhaled air

(71) Applicants: Stephen Cox, Lexington, KY (US); Geoffrey Charles Wolf, Tannay (CH)

(72) Inventors: Stephen Cox, Lexington, KY (US); Geoffrey Charles Wolf, Tannay (CH)

(21) Appl. No.: 14/499,769

(22) Filed: Sep. 29, 2014

(51) Int. Cl. A61M 16/22; A61M 16/20

(52) U.S. Cl. CPC A61M 16/22; A61M 16/20; A61M 2205/75; A61M 2202/0225

(57) ABSTRACT

Anxiety attacks can often be limited or eliminated by reducing the concentration of inhaled carbon dioxide. Disclosed is a handheld device for reducing the carbon dioxide concentration of inhaled air by using stacked sheets having rows of adsorbents deposited thereon and which form a removable cartridge. The cartridge is placed into an adsorbent cartridge cradle to secure the cartridge within the device housing and to maintain ventilation channels through the cartridge. Inhaled air passes through the adsorbent containing cartridge and exhaled air exits the device through a bypass to avoid poisoning the adsorbent with exhaled carbon dioxide.

### TECHNICAL FIELD

The present invention relates to a portable, handheld chemical adsorbent system/device for reducing anxiety and the symptoms of panic attacks through the reduction of inhaled carbon dioxide.

### BACKGROUND

Anxiety is a term used generally to describe several disorders whose symptoms include apprehension, fear, and nervousness either as an abnormal response to an environmental condition or sometimes without a precursor stressor. Mild anxiety is vague and unsettling, while severe anxiety can be extremely debilitating, having a serious impact on daily life.

The cause of this condition is still not understood, but it has been long known that the vulnerability to panic disorder is strongly genetic. The amygdala has a significant role in the experience of both instinctive fear and fear that is learned from life experiences. Studies have shown that that inhaling elevated concentrations of carbon dioxide can generally induce anxiety and trigger panic attacks. Some anxiety and panic reactions are known to require the presence of the acid-sensing protein in the amygdala. An increase in carbon dioxide in the bloodstream reduces blood pH. One of the most consistent research findings about patients suffering from panic disorder is that they are hypersensitive to carbon dioxide levels in the air and other precursors to increased brain acidity. Research indicates that 80% of patients with panic disorder typically experience a panic attack when they inhale air containing 5% CO₂ and that 2 breaths of 50% CO₂ can trigger attacks immediately within this group. Further research has shown that the close relatives of panic prone patients will also panic during carbon dioxide inhalation, despite having been previously asymptomatic for an anxiety disorder. A hypersensitivity to acid in the brain seems to be indicative of a predisposition to panic attacks. Research has shown that CO₂ levels of 600-950 ppm is commonly found in moderately populated enclosed areas (Cox, S, Lawrence, J and Sheehan, D, (1995). Single Ion Gas Chromatographic/Mass Spectroscopic Quantitative Analysis os Environmental CO₂ in Agoraphobic Environments; Anxiety 1; 275-7).

Generalized Anxiety Disorder, i.e. GAD, is a chronic disorder characterized by excessive, persistent anxiety about nonspecific life events, objects, and situations and GAD is diagnosed when a person worries excessively about a variety of everyday problems for at least 6 months. GAD sufferers often feel afraid and worry about their health, money, family, work, or school, but they have trouble both identifying the specific fear and controlling the worries. Their fear is usually unrealistic or out of proportion with what may be expected in their situation. Sufferers expect failure and disaster to the point that it interferes with daily functions like work, school, social activities, and relationships.

A panic attack is a sudden episode of intense fear that triggers severe physical reactions when there is no real danger or apparent cause. Panic attacks and anxiety are complex conditions for which little is known about their physiological triggers. It is believed that some individuals possess a hypersensitivity to elevated CO₂ levels that should normally be tolerated by healthy individuals.

The removal of CO₂ from air typically relies upon a chemical adsorbent to remove the undesired constituent from air and is commonly referred to as CO₂ scrubbing. Porous inorganic metal oxides such as alkali metals or alkali-earth metals providing alkalinity have also been widely incorporated for CO₂ capture. Among the various metal oxides, lithium and calcium based materials are preferred as effective CO₂ adsorbents because of their high adsorption capacity.

Other methods for CO₂ scrubbing techniques include chemical absorption through alkanolamine-based absorbents, ionic liquid-based absorbents, and blended absorbents. Additional methods incorporate physical adsorbents such as carbonaceous materials, e.g. activated carbon and graphene, zeolite, ordered mesoporous silica, e.g. M41s, SBA-n, and AMS and metal-organic frameworks. Chemical adsorbents include lithium materials, e.g. LiOH and Li₂O₂, calcium materials, e.g. Ca(OH)₂, and amine-based materials.

### SUMMARY

The subject device of the present application is a handheld scrubber utilized to remove undesired constituents from air prior to inhalation. The device is primarily utilized to reduce the concentration of CO₂ in inhaled air so as to inhibit the onset of anxiety or to provide a fast-acting intervention during the onset of anxiety or panic attacks among those afflicted with a hypersensitivity to CO₂.

A disproportionate tracheal/blood CO₂ ratio may induce a signal to the locus ceruleus-amygdala alarm and fear centers of the brain resulting in anxiety or even panic attacks in persons predisposed to such afflictions. Higher CO₂ levels have also been shown to impair the quality of higher order brain functions in persons without these afflictions. The desire to escape an environment detected to be rich in CO₂ is believed to be a physiological response common in mammals.

The hypersensitive response to elevated CO₂ levels is rapidly reversible once the CO₂ concentration falls below that required to induce the response. However, it is not always possible to remove a person so afflicted from a confined environment, e.g. a moving car, poorly ventilated work space or classroom, a crowded elevator, or an airplane. Additionally, time may be of the essence and the afflicted person may need to rapidly overcome the effect to focus on the task or decision at hand.

In order to provide an effective, rapid, and non-pharmacological treatment of symptoms related to the exposure to elevated CO₂ levels, a handheld air scrubber that can be carried in a pocket, briefcase, or purse has been developed which employs a chemical adsorbent to remove substantial quantities of CO₂ from air inhaled through the device until the adsorbent bed contained therein is substantially depleted. Ideally the device possesses valves to direct inhaled air across the adsorbent bed and to provide an alternative channel for exhaled air so as to direct CO₂ rich expirations out of the device without passing back across the adsorbent bed. Other undesired constituents may also be removed from air by selecting the appropriate absorbents and/or adsorbents.

### BRIEF DESCRIPTION OF THE DRAWINGS

FIG. 1 depicts a perspective view of a preferred embodiment of the handheld air scrubber.

FIG. 2 depicts a perspective view of a preferred embodiment of the filter cartridge housing.

FIG. 3 depicts an exploded perspective view of a preferred embodiment of the handheld air scrubber, filter cartridge housing, and filter cartridge.

FIG. 4 depicts a cross sectional view of a preferred embodiment of the handheld air scrubber along the proximal-distal (A-A′) axis.

FIG. 5 depicts a perspective view of the front of a preferred embodiment of the handheld air scrubber.

FIG. 6 depicts a partial cross sectional perspective view of the rear of a preferred embodiment of the handheld air scrubber depicting the adsorbent cartridge disposed therein.

FIG. 7 summarizes research results into the effect of elevated CO₂ levels on human decision making.

FIG. 8 depicts a cross-sectional view of the adsorbent cartridge.

FIG. 9 depicts a cross-sectional view of a single adsorbent sheet.

### DETAILED DESCRIPTION OF THE PREFERRED EMBODIMENTS

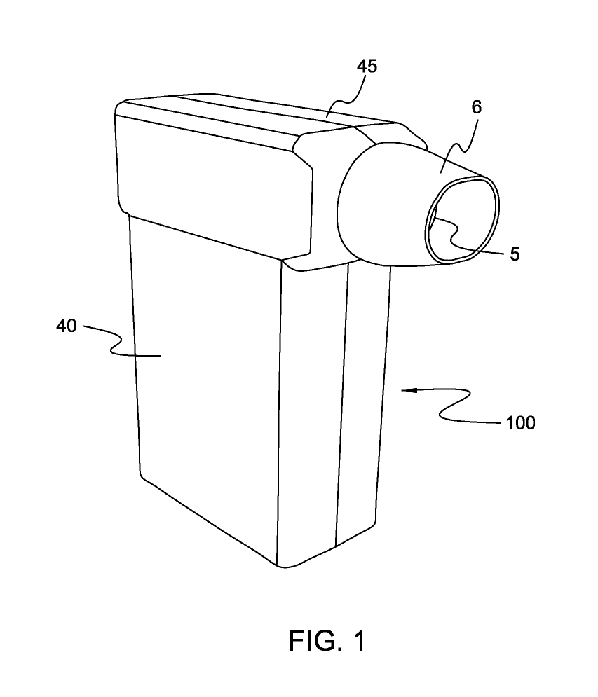

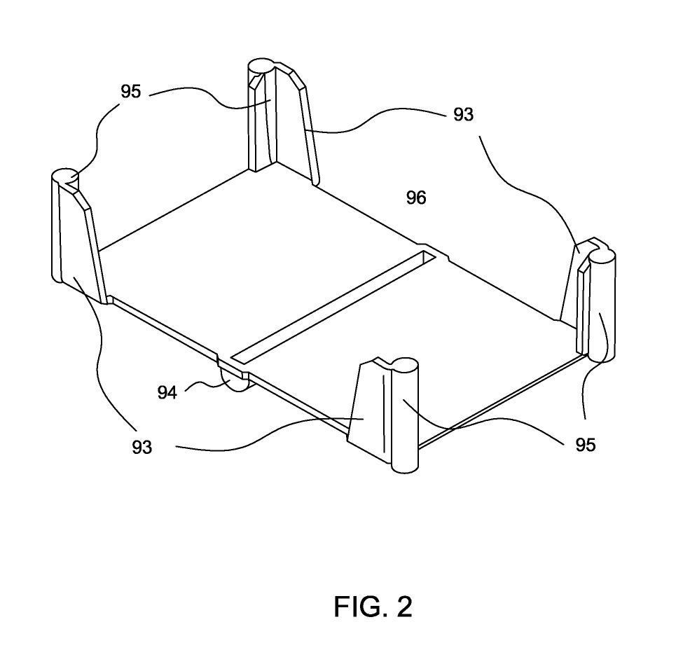

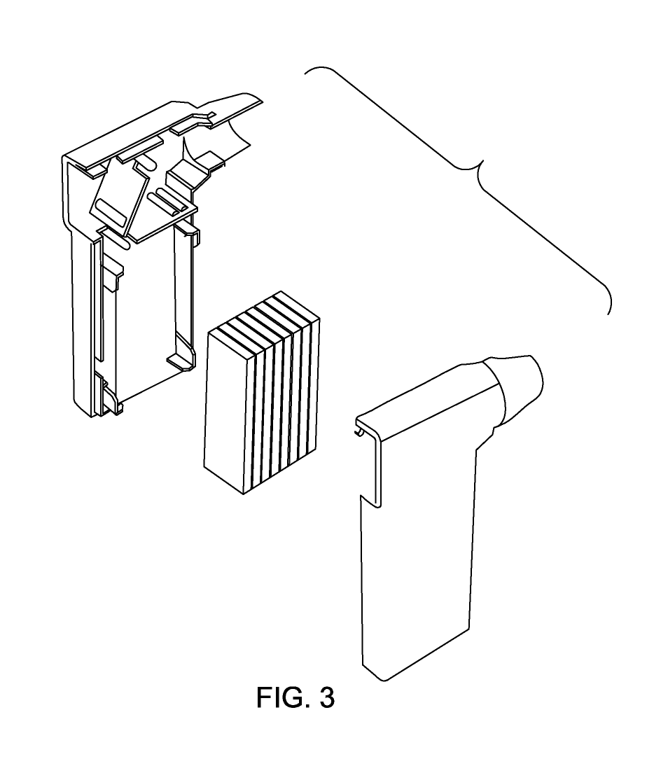

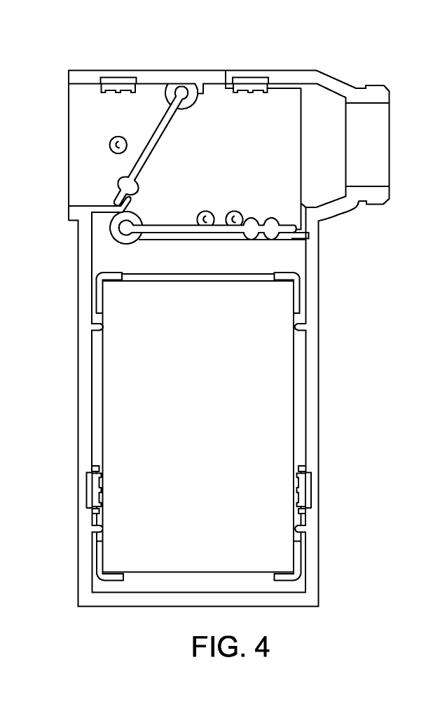

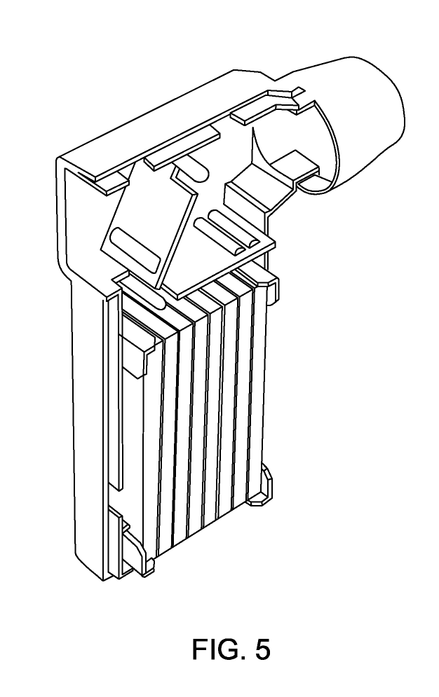

A handheld air scrubber device 100 for short term use is depicted in FIGS. 1-6 and 8-9. In a preferred embodiment, the handheld air scrubber device 100 possesses a housing 50 comprised of a first lateral casing 40 and a second lateral casing 45 which are joined at the casing joint 47.

The present application is directed to the disclosure of a handheld device 100 utilized to remove undesired constituents from ambient air immediately prior to its inhalation. This device is particularly useful in enclosed environments where these undesired constituents, e.g. CO₂ and CO, can accumulate to produce deleterious effects on humans who inhale such contaminants.

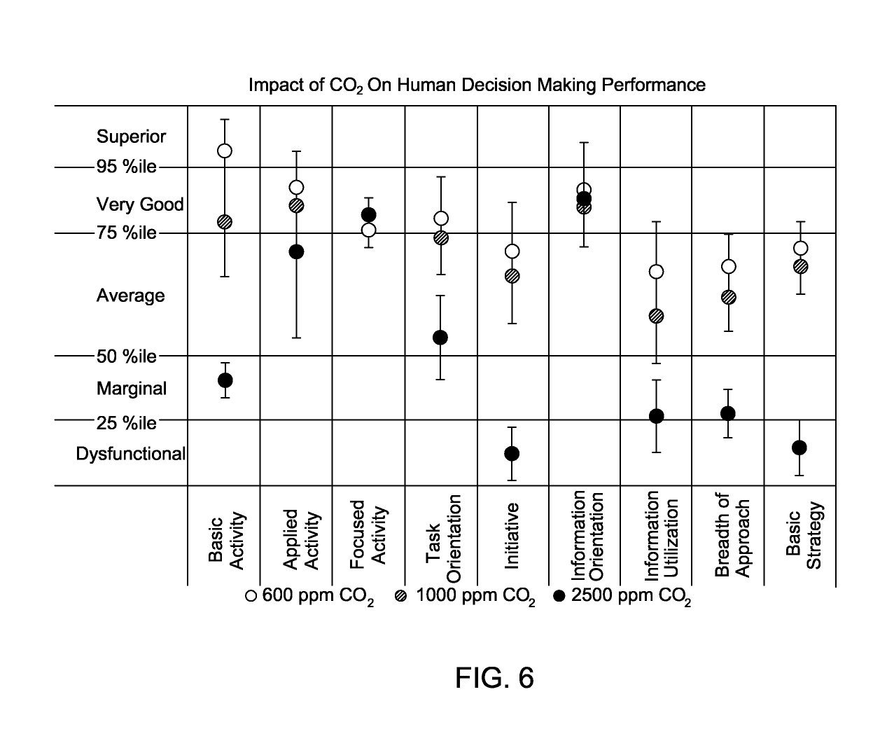

The process of purifying air is commonly known as “scrubbing.” Scrubbing CO₂ from inhaled air is useful to treat the onset of anxiety and panic attacks. Moreover, as is shown in FIG. 6, exposure to elevated levels of indoor CO₂ has been shown to impair human decision making. The use of the disclosed device is believed to not only alleviate some symptoms of the onset of anxiety and panic attacks, but is also believed to improve decision making by reducing the concentration of CO₂ in inhaled air.

A common method for scrubbing is through the use of chemical adsorbents 80 to remove undesired constituents as air comes into contact with a sufficient quantity of that adsorbent 80. The efficiency of the scrubbing process depends on the duration a given volume of air is in contact with an unsaturated adsorbent 80, i.e. residence time, the efficiency of the adsorbent 80 itself in removing the undesired constituent irrespective of time, and the rate of removal of the undesired constituent from air when in contact with the adsorbent 80.

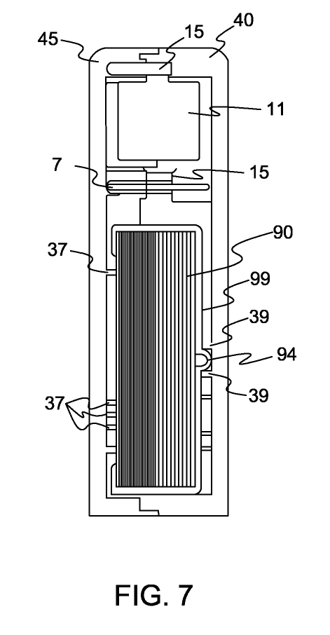

In a preferred embodiment, as depicted in FIG. 7, the handheld air scrubber 100 possesses a ventilation port 5 at the distal end of ventilation port sheath 6 that is placed in the mouth of the user and through which air is inhaled from the housing 50 along the inhalation route 60, and exhaled through the device 100 along the exhalation route 70. Inhaled air is drawn into the housing 50 through the inhalation port 17 as the user inhales and creates a vacuum. In a preferred embodiment, a plurality of inhalation ports 17 are utilized at the housing base 43. The inhaled air enters the air treatment chamber 30 as it passes through the inhalation ports 17 at the housing base 43. The air treatment chamber 30 houses the air treatment device 88 which acts to scrub undesired constituents from the air. In a preferred embodiment, the air treatment device 100 housed within the air treatment chamber 30 is an adsorbent cartridge 90.

The air treatment chamber 30 further possesses a means to secure the adsorbent cartridge 90 in a fixed position within the housing 50. In a preferred embodiment, the air treatment chamber 30 possesses cartridge ribs 37 extending from the first air treatment chamber wall interior surface 36 of the first air treatment chamber wall 48 and the second air treatment chamber wall interior surface 38 of the second air treatment chamber wall 49 of the lateral casings 40, 45 to mechanically hold the cartridge 90 in place in a friction fit arrangement. The air treatment chamber 30 further possesses base cartridge stops 58 extending into the air treatment chamber 30 so as to lift the cartridge 90 off of the inhalation ports 17. The air treatment chamber 30 further possesses a top cartridge stop 59 extending into the air treatment chamber 30 from the base 43 so as to set an upper limit for the movement of the cartridge 90 within the air treatment chamber 30.

The inhaled air is drawn through the inhalation ports 17 and through the cartridge 90 within the air treatment chamber 30. The air treatment chamber 30 is sealed at the top by a chamber ceiling 32. An internal inlet port 27 connects the air treatment chamber 30 to the transverse air duct 20 running from the anterior face 42 to the posterior face 46 of the device 100. An inlet valve 7 opens and closes the internal inlet port 27 by rotating above the inlet valve 7 and up and distally through the transverse air duct 20 about an inlet valve hinge 8 sited at the distal end of the internal inlet port 27. In a preferred embodiment, the inlet valve 7 is a flapper type valve with dowels 55 which are received into dowel seats 57 within the housing 50.

The inlet valve 7 rests on an inlet valve stop 9 which acts to prevent the inlet valve 7 from rotating into the air treatment chamber 30. At rest the inlet valve 7 seats against and seals the internal inlet port 27.

The outlet valve 11 rests on an outlet valve stop 12 which acts to prevent the outlet valve 11 from rotating proximally into the transverse air duct 20 and over the inlet valve 7. The outlet valve 11 acts to seal the outlet port 25 during inhalation. Sealing the outlet port 25 during inhalation ensures that a sufficient vacuum is created to draw air into the transverse air duct 20 from the air treatment chamber 30 and open the inlet valve 7.

In a preferred embodiment, the outlet valve 11 is a flapper type valve 53 with dowels 55 which are received into dowel seats 57 within the housing 50. At rest, the outlet valve 11 seats against the outlet port 25 within the transverse air duct 20 and prevents air from being inhaled through the exhaust port, bypassing the adsorbent cartridge 90.

During exhalation, this seals the air treatment chamber 30 and directs exhaled air through the transverse air duct 20 and against the outlet valve 11. The pressure of the exhaled air forces the outlet valve 11 open and allows exhaled air to exit the housing 50 through the exhaust port 13. Exhaled air is prevented from entering the air treatment chamber 30 and traversing the adsorbent cartridge 90 to avoid premature depletion of the adsorbent 80 by unnecessary exposure to constituents of the exhaled air.

In a preferred embodiment, the inlet valve 7 and outlet valve 11 are flapper type valves which rotate about hinges 15 at the base of each valve 7, 11. At rest, the inlet valve 7 lies closed against the top of the air treatment chamber 30 so as to seal the internal inlet port 27.

During inhalation, the inlet valve 7 opens due to the vacuum within the transverse air duct 20 and permits air to flow from the inhalation ports 17 across the cartridge 90 in the air treatment chamber 30, through the inlet valve 7, and into the transverse air duct 20. The vacuum also acts to close the outlet valve 11 which seals the outlet port 25. Inhaled air follows the inhalation path 60 and exits the ventilation port 5 into the mouth of the user via the ventilation port sheath 6.

During exhalation, air is forced into the housing through the ventilation port 5, across the transverse air duct 20, against the outlet valve 11 which is hingedly attached at the top of the exhaust port 13 in the transverse air duct 20. The force of the air exhaled into the housing 50 via the ventilation port 5 forces the inlet valve 7 to close and opens the outlet valve 11. After passing through the outlet port 25, the exhaled air exits through the exhaust port 13 at the distal end of the transverse air duct 20.

The housing 50 is preferably constructed of a rigid material such as a plastic to protect the integrity of the adsorbent sheets. The inlet valve 7 and outlet valve 11 are likewise constructed of a rigid material. The housing 50 is preferably molded into two lateral casings 40, 45 which are joined about a casing joint 47.

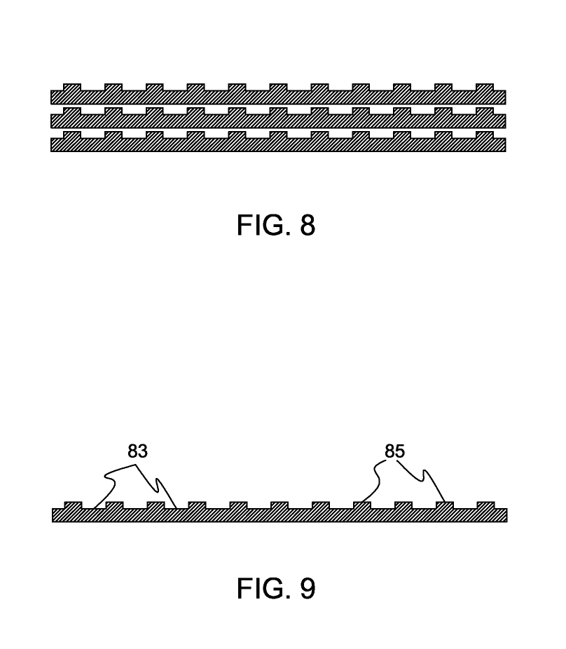

As depicted in FIGS. 7-8, the adsorbent 80 is preferably retained on a planar adsorbent support 31. Dustless preparations of commercially available adsorbents are preferred so as to avoid issues with the inhalation of adsorbent 80. Dustless adsorbents 80 are especially preferred when alkali metals and alkali earth metals containing adsorbents 80 are utilized due to the potential for chemical burns or when toxic adsorbents 80 are employed. In a preferred embodiment, the adsorbent support 31 is constructed of a card stock or other sufficiently rigid cellulosic or similar material with sufficient rigidity to maintain its form but pliable so as to permit the cartridge ribs 37 in the air treatment chamber 30 to deform and hold the edges of the filter cartridge housing 99.

In a further preferred embodiment, the adsorbent 80 is retained on each support 31 in parallel vertical rows 85 across one face of the support 31. When stacked together, these supports 31 are arranged so that the adsorbent rows 85 on each support 31 lie adjacent to the planar surface of an adjacent support 31. This arrangement, when supports 31 are tightly stacked together to form an adsorbent cartridge 90, uses the empty rows created from the open spaces between adsorbent rows 85 as adsorbent cartridge ventilation channels 83 through which air is inhaled and contacts the adsorbent 80 for the removal of CO₂ or other contaminants. Ideally, there is sufficient volume in the cartridge ventilation channels 83 to minimize the pressure differential across the cartridge 90.

The thickness of the adsorbent cartridge 90 is optimized to ensure a tight fit within the air treatment chamber 30 so as to ensure sufficient air flow through the adsorbent cartridge 90 rather than around the adsorbent cartridge 90. Ideally the supports 31 are bound together to fix each sheet's relative position within the adsorbent cartridge 90. The air treatment chamber 30 utilizes top cartridge stops 59 to ensure that a head space is preserved by preventing the adsorbent cartridge 90 from moving up toward the internal inlet port 27. The air treatment chamber 30 also possesses base cartridge stops 58 to ensure that some space is maintained between the bottom of the adsorbent cartridge 90 and the air treatment chamber floor 35.

The air treatment chamber floor 35 possesses a plurality of inhalation ports 17 through which air enters the housing 50 during inhalation. Some space between the bottom of the adsorbent cartridge 90 and the inhalation ports 17 assists in inhibiting foreign objects from contacting the adsorbent supports and adsorbent rows in a way that could potentially dislodge some adsorbent 80 from the supports 31.

The adsorbent cartridge housing 99, i.e. filter cartridge cradle 99, retains the filter cartridge 90, i.e. adsorbent cartridge 90. The adsorbent cartridge housing 90 possesses a substantially rectangular support floor 96 from which support stops 95 extend vertically from each corner on the proximal side of said support floor 96 along with side cartridge guides 93 and end cartridge guides 97 which serves to retain and position the adsorbent 36

cartridge 90. It is important to keep the absorbent sheets 91 properly aligned to keep the filter ventilation channels 83 open and unobstructed and to minimize the pressure differential (AP) across the cartridge 90. When properly positioned, the ventilation channels 83 minimize the pressure differential by keeping all or most of the channels 83 open and contiguous across the cartridge 90. While it is important to keep the sheets 91 properly aligned along their edges, it is also important to prevent them from moving along the proximal-distal axis, i.e. up-and-down within the cartridge cradle 99 as they could shift and cause channels 83 to be blocked.

The distal side of said support floor 96 possesses at least one tab 94 extending vertically down and away from said support floor 96 to contact the air treatment chamber first side wall 49 on one side of the air treatment chamber 30 which acts to tension the filter cartridge cradle 99 and filter cartridge 90 against a second air treatment chamber wall 48. The tab 94 contacts the first side wall 49 between the tab positioning ridges 39 which serve to hold the filter cartridge cradle 99 in place.

It is anticipated that the device 100 will be disposable, with the housing 50 being supplied pre-loaded with an adsorbent cartridge 90. The device 100 is further anticipated to be sold in sealed packaging. The packaging could be vacuum sealed or possess an inert atmosphere such as N₂.

While the present invention may have been disclosed herein with reference to certain embodiments, it will be apparent that modifications and variations are possible without departing from the spirit and scope of the invention as defined herein. Furthermore, it should be appreciated that any and all examples in the present disclosure, while illustrating embodiments of the invention, are provided as non-limiting examples and are, therefore, not to be taken as limiting the various aspects so illustrated. The present invention is intended to have its full scope consistent with the drawings and description herein, and equivalents thereof. Accordingly, the drawings and detailed description are to be regarded as illustrative and not as restrictive.

What is claimed is:

1. A device for removing undesired constituents from air, said device comprising:
a housing, said housing having a ventilation port for insertion into the mouth through which air is inhaled from said housing and exhaled into said housing;
an exhaust port in said housing through which exhaled air is expelled from the housing;
an air inlet port in said housing through which inhaled air is drawn into and through said housing to said ventilation port along an inhalation channel which directs air from said air inlet port to said ventilation port through a series of walls and directional valves;
an air treatment device arranged within said inhalation channel;
at least one air inlet valve, said air inlet valve arranged to open upon inhalation to direct air flow through said air inlet port and along said inhalation channel to exit through said ventilation port and close upon exhalation through said ventilation port into said housing and along an exhalation channel so as to inhibit exhaled air from entering said air treatment chamber within said housing;
at least one air outlet valve, said air outlet valve arranged to open upon exhalation to direct air flow entering said housing through said ventilation through said housing to exit through said exhaust port and close upon inhalation through said ventilation port into said housing so as to inhibit the introduction of air into said housing from said exhaust port; and
a removable housing for aligning and positioning said air treatment device within said inhalation channel.

2. The device of claim 1, wherein said air treatment device possesses an adsorbent for the removal of undesirable constituents from air inhaled through said ventilation port.

3. The device of claim 2, wherein said adsorbent is retained on an adsorbent support.

4. The device of claim 3, wherein said adsorbent support is a planar sheet.

5. The device of claim 4, wherein said housing possesses an air treatment chamber to house said adsorbent support.

6. The device of claim 4, wherein an adsorbent support possesses means to mechanically separate adjacent support surfaces so as to permit the flow of air between said adjacent support surfaces by establishing a minimum distance between two said adjacent support surfaces.

7. The device of claim 6, wherein said means to mechanically separate support surfaces is a plurality of parallel rows on at least one surface of each said support.

8. The device of claim 7, wherein said rows are at least partially comprised of said adsorbent.

9. The device of claim 8, wherein said adsorbent support is a substantially planar substrate.

10. The device of claim 9, wherein a plurality of said substrates are arranged in a substantially fixed spatial relationship relative to each and with said rows being vertically oriented between substrates.

11. The device of claim 10, wherein said substrates are fastened together to form an adsorbent cartridge.

12. The device of claim 2, wherein said undesirable constituent is CO₂.

13. The device of claim 12, wherein said air conditioned by said air treatment device reduces the relative volumetric concentration of CO₂ of air by at least 50% for at least 100 typical human respirations.

14. The device of claim 13, wherein said air conditioned by said air treatment device reduces the relative volumetric concentration of CO₂ of air by at least 60% for at least 100 typical human respirations.

15. The device of claim 14, wherein said air conditioned by said air treatment device reduces the relative volumetric concentration of CO₂ of air by at least 70% for at least 100 typical human respirations.

16. The device of claim 2, wherein said undesirable constituent is selected from the group consisting of CO, H₂O, NOx, and SOx.

17. The device of claim 2, wherein said undesirable constituent is a volatile organic compound.

18. The device of claim 2, wherein said undesirable constituent is selected from the group consisting of acids and bases.

19. The device of claim 2, wherein said undesirable constituent is selected from the group consisting of chemical and biological toxins.

20. The device of claim 2, wherein said adsorbent is selected from the group consisting of LiOH and Ca(OH)₂.

21. A device for removing undesired constituents from air, said device comprising:
a housing, said housing having a ventilation port for insertion into the mouth through which air is inhaled from said housing and exhaled into said housing;
an exhaust port in said housing through which exhaled air is expelled from the housing;
an air inlet port in said housing through which inhaled air is drawn into and through said housing to said ventilation port along an inhalation channel which directs air from said air inlet port to said ventilation port through a series of walls and directional valves;
an air treatment device arranged within said inhalation channel, said air treatment device containing an adsorbent for the removal of undesirable constituents from air inhaled across said adsorbent;
at least one air inlet valve, said air inlet valve arranged to open upon inhalation to direct air flow through said air inlet port and along said inhalation channel to exit through said ventilation port and close upon exhalation through said ventilation port into said housing and along an exhalation channel so as to inhibit exhaled air from entering said air treatment chamber within said housing;
at least one air outlet valve, said air outlet valve arranged to open upon exhalation to direct air flow entering said housing through said ventilation through said housing to exist through said exhaust port and close upon inhalation through said ventilation port into said housing so as to inhibit the introduction of air into said housing from said exhaust port; and
a removable housing for aligning and positioning said air treatment device within said inhalation channel.

22. The device of claim 21, wherein said adsorbent is sited in a plurality of parallel rows on a planar adsorbent support housed within an air treatment chamber through which air is inhaled.

23. The device of claim 22, wherein a plurality of planar of adsorbent supports are bound together as an adsorbent cartridge.

24. The device of claim 23, wherein the undesirable constituent is CO₂ and the adsorbent is selected from the group consisting of LiOH and Ca(OH)₂.

25. A device for removing undesired constituents from air, said device comprising:
a housing, said housing having a ventilation port for insertion into the mouth through which air is inhaled from said housing and exhaled into said housing;
an exhaust port in said housing through which exhaled air is expelled from the housing;
an air inlet port in said housing through which inhaled air is drawn into and through said housing to said ventilation port along an inhalation channel which directs air from said air inlet port to said ventilation port through a series of walls and directional valves;
an air treatment device arranged within said inhalation channel, said air treatment device having a plurality of sheets on which an adsorbent for the removal of CO₂ from air is deposited, said air treatment device placed within said inhalation channel between said air inlet port and said ventilation port;
at least one air inlet valve, said air inlet valve arranged to open upon inhalation to direct air flow through said air inlet port and along said inhalation channel to exit through said ventilation port and close upon exhalation through said ventilation port into said housing and along an exhalation channel so as to inhibit exhaled air from entering said air treatment chamber within said housing; and
at least one air outlet valve, said air outlet valve arranged to open upon exhalation to direct air flow entering said housing through said ventilation through said housing to exit through said exhaust port and close upon inhalation through said ventilation port into said housing so as to inhibit the introduction of air into said housing from said exhaust port; and
a removable housing for aligning and positioning said air treatment device within said inhalation channel.

26. The device of claim 25, wherein said adsorbent is retained in a plurality of parallel rows on a planar adsorbent support housed within an air treatment chamber through which air is inhaled.

27. The device of claim 26, wherein a plurality of planar adsorbent supports are bound together as an adsorbent cartridge.

28. The device of claim 27, wherein the adsorbent is selected from the group consisting of LiOH and Ca(OH)₂.
